(19) United States
(12) Patent Application Publication
(10) Pub. No.: US 2025/0064881 A1
(43) Pub. Date: Feb. 27, 2025

(54) TOPICAL COMPOSITION AND APPLICATOR THEREFOR FOR RELIEVING CHALAZION

(71) Applicant: LABORATOIRES THEA, CLERMONT-FERRAND (FR)

(72) Inventor: Luc LEBRETON, BEAUMONT (FR)

(21) Appl. No.: 18/723,807

(22) PCT Filed: Dec. 23, 2022
(86) PCT No.: PCT/EP2022/087784

§ 371 Date: Jun. 24, 2024

(30) Foreign Application Priority Data
Dec. 23, 2021 (FR) FR2114348

(51) Int. Cl. A61K 36/47; A61K 9/00; A61K 35/06; A61K 36/889; A61K 47/14

(52) U.S. Cl. CPC A61K 36/47; A61K 9/0014; A61K 35/06; A61K 36/889; A61K 47/14

(57) ABSTRACT

A topical composition having
castor oil;
coconut oil, advantageously refined coconut oil, jojoba oil, or avocado oil;
vaseline;
and to its administration using a roll-on applicator for the treatment in particular of chalazion.

### TECHNICAL FIELD

The present invention relates to the field of dermatological care related to eye health, more particularly to the treatment of swelling present in the eyelid. More specifically, the invention relates to a topical composition for the treatment of chalazion.

### PRIOR ART

Several pathologies, such as chalazion, stye or blepharitis, are characterized by the appearance of localized swelling in the eyelid. This swelling results in several unpleasant symptoms, such as puffiness, a bump, redness, tingling, a burning sensation on the eyelid, or even watery eyes, or the formation of dandruff on the eyelashes.

The origin of the swelling of the eyelid is variable and allows the pathology to be qualified according to its origin:

Blepharitis is a skin inflammation of the eyelid, caused by an infection or allergy, responsible for the swelling.

A stye is an infection, particularly bacterial, of the meibomian gland located in the thickness of the eyelid, responsible for the swelling.

Chalazion is caused by a non-infectious occlusion of the meibomian glands, responsible for the inflammatory swelling.

Thus, the cause of chalazion is not of infectious origin, unlike blepharitis and stye.

In practice, the meibomian glands (tarsal glands) are sebaceous glands located in the epidermis of the eyelids. More precisely, they are located in the cartilage of the eyelids, 30 to 40 per eyelid, oriented perpendicular to the free edge protected by the eyelashes. They secrete a fatty substance of sebum type, which generally mixes with tears to lubricate the surface of the eye.

In the event that they do not function correctly, particularly in the case of chalazion, an excessive accumulation of secretions from the meibomian glands causes them to become clogged, particularly at their evacuation orifice located on the free edge of the eyelid. This clogging results in an enlargement of the gland, in the form of a cyst or inflammatory granuloma. The chalazion can be internal or external. The external chalazion causes thickening of the eyelid, visible on the eyelid. The eyelid may thus be red and swollen. The internal chalazion causes lacrimation and slight edema under the eyelid.

Common, benign and not serious, the chalazion, which has no infectious cause, can be reabsorbed in several weeks.

However, depending on its size and location, the chalazion can become bothersome, painful, or even indent the cornea and/or cause blurred vision. If it persists, the chalazion must be removed surgically, by making a conjunctival incision in the gland under local anesthesia.

Before the surgical procedure and as soon as the chalazion appears, it is possible to apply warm moist compresses daily (2 to 3 times a day) to reabsorb the chalazion. It is also known to apply an ointment based on an antibiotic and/or cortisone to chalazion.

The document “11 Home Remedies to Treat Chalazion in Children” (2018 Apr. 23; URL: https://parentinghealthybabies.com/chalazion-children-home-remedies/) describes various products, including castor oil or coconut oil, that can be used at home to treat chalazion.

However, these treatments have limited effectiveness: while they can temporarily alleviate symptoms, it is not uncommon for the chalazion to return.

In addition, their method of application is often complicated and inconvenient for the patient.

Thus, applying a hot compress several times a day can be inconvenient: it requires a hot water source and is time-consuming (5 to 10 minutes of application).

Applying an antibiotic and/or cortisone ointment directly to the eyelid with a finger can be painful in the chalazion area and can cause infectious complications due to contamination if the hands have not been properly disinfected. In addition, manual application of an ointment does not allow precise control of the dose deposited or of the targeted area, which could lead to unwanted penetration of the ointment into the eye.

Thus, there is a clear need to develop new solutions to treat chalazion, which do not have the aforementioned drawbacks.

### DETAILED DESCRIPTION OF THE INVENTION

The invention relates to a topical composition comprising judiciously chosen ingredients to be combined with a massaging motion, advantageously using a “roll-on” type application device.

As demonstrated in the present application, this constitutes a particularly suitable solution for the management of chalazion, resulting in a reduction in irritation, redness and swelling of the eyelid and therefore improved comfort for the patient.

### Definitions

The definitions below give the meaning generally used in the context of the invention and must be taken into account unless another definition is explicitly indicated.

In the context of the invention, the articles “a” and “an” are used to designate one or more (that is to say at least one) of the grammatical objects of the article. For example, “an element” designates at least one element, that is to say one or more elements.

The terms “roughly”, “about,” “on the order of” or “approximately” used in this document to refer to a measurable value such as a quantity, a duration, etc., should be understood to include variations of ±20% or ±10%, preferably ±5%, more preferably ±1%, and even more advantageously ±0.1% with respect to the specified value.

Intervals/ranges: Throughout this disclosure, various aspects of the invention may be presented in the form of an interval of values (range format). It should be understood that the description of values in the form of an interval is made only for convenience and brevity and should not be construed as a limitation of the scope of the invention. Accordingly, the description of a range must be considered to have specifically disclosed all the possible subranges as well as the individual numerical values within that range. For example, the description of a range such as “from 1 to 6” should be considered to have specifically disclosed subranges such as from 1 to 3, from 1 to 4, from 1 to 5, from 2 to 4, from 2 to 6, from 3 to 6, etc. as well as individual numbers within this range, for example 1, 2, 2.7, 3, 4, 5, 5.3 and 6. This applies regardless of the extent of the range.

The term “abnormal”, when used in the context of organisms, tissues, cells or components thereof, means organisms, tissues, cells or components thereof that differ in at least one observable or detectable characteristic (for example, age, treatment, time of day, etc.) from organisms, tissues, cells or components thereof that exhibit the respective “normal” (expected) characteristic. Characteristics that are normal or expected for one cell or tissue type may be abnormal for another cell or tissue type.

The terms “patient,” “subject,” “individual” etc. are used interchangeably in this document and refer to any animal or cell thereof, in vitro or in situ, which may be subjected to the methods described in this document. In certain non-limiting embodiments, the patient, subject or individual is an animal, preferably a mammal, more advantageously a human.

Within the meaning of the invention, a “disease” or “pathology” is a state of health of an animal in which its homeostasis is negatively affected and which, if the disease is not treated, continues to deteriorate. Conversely, within the meaning of the invention, a “disorder” or “dysfunction” is a state of health in which the animal is capable of maintaining its homeostasis but in which the state of health of the animal is less favorable than it would be in the absence of the disorder. If left untreated, a disorder does not necessarily lead to a deterioration in the animal's state of health over time.

A disease or disorder is “alleviated” (“reduced”) or “improved” if the severity of a symptom of the disease or disorder, the frequency with which that symptom is experienced by the subject, or both, is/are reduced. This also includes the disappearance of disease progression, that is to say stopping the progression of the disease or disorder. A disease or disorder is “cured” (“recovered”) if the severity of a symptom of the disease or disorder, the frequency with which a symptom is experienced by the patient, or both, is/are eliminated.

In the context of the invention, a “therapeutic” treatment is a treatment administered to a subject who presents the symptoms (signs) of a pathology, with the aim of reducing or eliminating these symptoms. In the context of the invention, “treatment of a disease or disorder” means reducing the frequency or severity of at least one sign or symptom of a disease or disorder experienced by the subject. A treatment is said to be prophylactic when it is administered to prevent the development, spread or worsening of a disease, particularly if the subject does not or does not yet show symptoms of the disease and/or for which the disease has not been diagnosed.

As used herein, “treating a disease or disorder” means reducing the frequency or severity of at least one sign or symptom of a disease or disorder experienced by a subject. Disease and disorder are used interchangeably in the context of the treatment according to the invention.

Within the meaning of the invention, an “effective quantity” of a compound is the quantity of compound which is sufficient to provide a beneficial effect to the subject to whom the compound is administered. The term “therapeutically effective quantity” refers to a quantity that is sufficient or effective to prevent or treat (in other words slow or stop the development, stop progression, inhibit, reduce, diminish or reverse) a disease or disorder, including relief of the symptoms of that disease or disorder.

In the remainder of the description, the percentages (%) correspond to weight percentages (also referred to as percentages by weight), unless otherwise specified. Thus the percentage of an ingredient corresponds to the weight of said ingredient relative to the total weight of the composition.

According to a first aspect, the present invention relates to a topical composition based on:
castor oil;
coconut, jojoba or avocado oil, advantageously coconut oil, even more advantageously refined coconut oil; and
vaseline.

Thus, a composition according to the invention comprises:
castor oil;
coconut oil, advantageously refined coconut oil, jojoba oil, or avocado oil; and
vaseline.

In other words, a composition according to the invention may comprise:
castor oil, coconut oil and vaseline; or
castor oil, refined coconut oil and vaseline; or
castor oil, jojoba oil and vaseline; or
castor oil, avocado oil and vaseline.

One of the ingredients of a composition according to the invention is castor oil.

Castor oil is a non-food vegetable oil. It is obtained from castor bean seeds (Ricinus communis) and appears in the form of a colorless or light yellow viscous liquid. The triglycerides, of which it consists, include, for approximately 90% of its fatty acids, a C18 fatty acid, ricinoleic acid (or (R)-(+)-12-hydroxy-9Z-octadecenoic acid) which is a hydroxylated omega-9 and forms ricinolein, the predominant triglyceride of this oil. Its use in cosmetics is largely intended for the care of hair, eyelashes and eyebrows.

According to a particular embodiment, the castor oil represents 40 to 60%, advantageously 45 to 55%, even more advantageously 50% by weight of the composition.

A second ingredient of a composition according to the invention is a vegetable oil such as coconut oil, advantageously refined coconut oil.

Coconut oil is available in two main forms, depending on how it is produced:
Virgin coconut oil is obtained by cold pressing the fresh endosperm of the coconut.
Refined coconut oil, known as copra oil, is obtained by hot pressing of the dried endosperm, then refined and deodorized. Very rich in saturated fatty acids, refined copra oil is used for its emollient properties.

It is used in cosmetics, particularly to nourish the skin and hair.

As is known, coconut oil is generally in solid form at a temperature below 25° C. For the preparation of the composition according to the invention, or even its use, it may be useful to heat this ingredient to a higher temperature, or even the composition or its application device as will be described below.

Alternatively, jojoba oil or avocado oil can be used.

According to a particular embodiment, such an oil represents 20 to 40%, advantageously 25 to 35%, even more advantageously 30% by weight of the composition.

Vaseline constitutes the third ingredient of a composition according to the invention.

Vaseline (or petroleum jelly) is a fatty substance of mineral origin, more precisely a petroleum distillate. It is essentially made up of alkanes, with the general formula (CₙH₂ₙ₊₂) and is fluid or even pasty at room temperature. Officinal vaseline is an ointment used as an adjunctive treatment for irritation, burns and dry skin lesions. Its most famous use is in the form used for lip care. It is also part of the composition of the majority of cosmetic lotions, used as excipient.

According to a particular embodiment, the vaseline represents 5 to 30% or even 5 to 20%, advantageously 10 to 15%, even more advantageously 12% by weight of the composition.

Thus, a composition according to the invention comprises at least 3 fatty substances. As is known, fatty substances can be of mineral, animal, vegetable or synthetic origin.

According to one embodiment, the composition according to the invention comprises the following 3 fatty substances:
a) castor oil;
b) coconut oil, advantageously refined coconut oil, jojoba oil or avocado oil; and
c) vaseline.

According to a particular embodiment, the composition according to the invention comprises only the 3 fatty substances mentioned above (a, b and c).

According to another particular embodiment, the composition according to the invention comprises only 2 fatty substances of plant origin, advantageously the 2 vegetable oils mentioned above (a and b).

According to another particular embodiment:
castor oil represents 40 to 60%, advantageously 45 to 55%, even more advantageously 50% by weight of the composition; and/or
coconut oil, advantageously refined coconut oil, represents 20 to 40%, advantageously 25 to 35%, even more advantageously 30% by weight of the composition; and/or
vaseline represents 5 to 30%, and even 5 to 20%, advantageously 10 to 15%, even more advantageously 12% by weight of the composition.

In the context of the present invention, it has been shown that the combination of these 3 ingredients, none of which is considered a therapeutic active ingredient strictly speaking, had a beneficial effect in the management of chalazion and that the combination of these 3 ingredients of interest was sufficient to achieve such an effect.

In the context of the invention, a therapeutic active ingredient (or active substance or active principle or active ingredient) is defined as a compound or substance having a pharmacological activity of its own recognized in connection with a given pathology, which is generally associated with the drug status and the obtention of a marketing authorization.

Thus and according to a first embodiment, the composition according to the invention does not contain any therapeutic agent, in particular for ophthalmological or dermatological purposes.

Alternatively, the composition according to the invention may contain a therapeutic agent intended for the treatment of the same pathology as those addressed in the present application or of a different pathology, advantageously ophthalmological or dermatological. Note that alternatively, such a therapeutic agent can be administered separately and therefore may not be contained in the composition according to the invention. Such administration can be carried out simultaneously or staggered over time.

Thus and in relation to chalazion, a corticosteroid (for example dexamethasone or hydrocortisone) and/or an antibiotic (for example azithromycin, neomycin, norfloxacin, oxytetracycline, ofloxacin or rifamycin) can also be administered, or even added to a composition according to the invention. For the treatment of a stye, a suitable antibiotic will be advantageously used.

According to a particular embodiment, the composition according to the invention does not contain selenium disulfide (SeS₂), glycerol monolaurate, caffeine and/or silk fibroin.

In addition and in a manner known to those skilled in the art, the composition of the invention may comprise, in addition to the 3 aforementioned ingredients of interest of fatty substance type, a pharmaceutically acceptable carrier. In a particular embodiment, the term “pharmaceutically acceptable” means approved by a federal or state regulatory agency or listed in the U.S. or European Pharmacopeia or another pharmacopeia generally recognized for use in animals and humans. The term “carrier” means a diluent, an adjuvant, an excipient or a vehicle with which the aforementioned products are administered.

According to a particular embodiment, the composition comprises at least one agent making it possible to stabilize and homogenize the mixture of the 3 ingredients of interest mentioned above of the fatty substance type that are present. In other words, one or more agents make it possible to improve the stability of this mixture of fatty substances and/or to improve the miscibility of these fatty substances with each other, in particular of castor oil and vaseline, to avoid phase separation over time of these fatty substances.

Such agents are typically thickeners, consistency agents or viscosity agents.

Such agents are, for example:
glyceryl stearate (for example, Kolliwax® GMS II from BASF);
glyceryl stearate (˜20%)+cottonseed oil (˜80%) (for example, GreenSoft® 330 Cotton from Greentech);
cetostearyl alcohol (for example, Lanette® O from BASF);
PEG sorbitan monooleate (for example, Tween® 80 from MERCK);
octyldodecanol (for example, octyldodecanol from Sigma Aldrich);
sorbitan laurate, sorbitan stearate, sorbitan oleate (for example, Montane™ 20, Montane™ 60, Montane™ 80, respectively from SEPPIC);
polyoxyl castor oil (for example, Kolliphor® RH40 from BASF);
glyceryl behenate (for example, COMPRITOL® 888 GATTEFOSSE pellets).
glycerol monostearate (for example GELEOL® MONO AND DIGLYCERIDES from GATTEFOSSE);
or mixtures thereof.

Thus and for a suitable formulation, the composition according to the invention may contain a viscosity agent such as glyceryl behenate (CAS number=77538-19-3). It may for example be the product Compritol® marketed by Gattefosse (COMPRITOL® 888 pellets). In the context of the invention, a viscosity agent aims to increase the viscosity of a composition.

In a manner known to those skilled in the art, the quantity of viscosity agent is adjusted according to the desired viscosity for the composition, as indicated below. According to a particular embodiment and in relation to glyceryl behenate, it advantageously represents 0.5 to 10%, advantageously 3 to 7%, even more advantageously 5% by weight of the composition.

In addition and for a suitable formulation, the composition according to the invention may contain an emulsifying agent such as glycerol or glyceryl monostearate (CAS number=85251-77-0 or 31566-31-1) corresponding to the glycerol ester of stearic acid. It may for example be the product Géléol® marketed by Gattefossé (GELEOL® MONO AND DIGLYCERIDES). In the context of the invention, an emulsifying agent aims to stabilize the composition.

In a manner known to those skilled in the art, the quantity of emulsifying agent is adjusted according to the desired behavior for the composition according to the invention. According to a particular embodiment and in relation to the glycerol monostearate, it represents advantageously 0.5 to 10%, advantageously 1 to 5%, even more advantageously 3% by weight of the composition.

Thus and according to a particular embodiment, the composition comprises:
castor oil;
coconut oil, advantageously refined coconut oil, jojoba oil, or avocado oil;
vaseline;
glyceryl behenate (Compritol®) and/or glycerol monostearate (Géléol®), advantageously glyceryl behenate (Compritol®) and glycerol monostearate (Géléol®).

According to another particular embodiment:
glyceryl behenate (Compritol®) represents 0.5 to 10%, advantageously 3 to 7%, even more advantageously 5% by weight of the composition; and/or
glycerol monostearate (Géléol®) represents 0.5 to 10%, advantageously 1 to 5%, even more advantageously 3% by weight of the composition.

In practice, glyceryl behenate (Compritol®) and glycerol monostearate (Géléol®) are also fatty substances.

Consequently and according to a particular embodiment, a composition according to the invention comprises the 3 fatty substances corresponding to the 3 ingredients of interest mentioned above, including 2 of plant origin, and 1 or 2 fatty substances, advantageously 2 fatty substances corresponding to pharmaceutically acceptable carriers, advantageously one or two agents making it possible to stabilize and homogenize the mixture of the 3 ingredients of interest.

According to a preferred embodiment, a composition according to the invention does not contain preservatives, in particular those commonly used in connection with cream and ointment type products to be applied to the skin.

In a preferred embodiment, the composition according to the invention is formulated in accordance with routine procedures for a composition suitable for topical administration in humans. The area targeted by the administration is advantageously the skin, and more particularly the eyelids. Advantageously, the mucous membranes, the conjunctiva and/or the cornea are not targeted.

According to a preferred embodiment, the composition according to the invention is in the form of a gel, an emulsion, an oil, a balm, a cream or an ointment, of homogeneous consistency and suitable for spreading in a wide range of temperatures, while avoiding its flow, particularly into the eyes and without leaving a greasy film on the targeted area.

According to one embodiment, the composition according to the invention is homogeneous and does not undergo phase separation between 4° C. and 40° C.

According to another embodiment, the composition according to the invention has a homogeneous consistency at a temperature between 20° C. and 30° C., typically at room temperature.

Advantageously, such a composition contains no aqueous phase or a reduced quantity of aqueous phase to maintain microbiological stability without adding a preservative.

Even more advantageously, this composition is compatible with administration using a “roll-on applicator” type device (also called “roll-on”).

In a manner known in this technical field, a “roll-on” is a receptacle or container, such as a tube or a bottle, whose outlet orifice is an advantageously rotating ball and which releases the composition it contains when there is contact between the ball and the surface to be treated. This release can be helped by slight pressure exerted on the container. In known manner, such a device makes it possible to preserve a composition and apply it locally, in a controlled manner (deposition of a determined quantity of the composition uncontaminated by contact with the fingers on a targeted area). In addition, it can be equipped with a cap or lid allowing the composition to be kept away from air, water and possible external contamination (pollutants, dust, etc.).

According to a particular embodiment, the receptacle, transparent or opaque, is made of plastic material, for example polyethylene (PE), advantageously high density polyethylene (HDPE) such as the Polyfoil® device marketed by Neopac. COEX tubes can also be used: these are coextruded polyethylene (PE) tubes lined with a barrier layer of ethylene vinyl alcohol (EVOH). It is advantageously flexible, that is to say it has the capacity to be deformed under the action of pressure exerted manually. The ball, for example 10 mm in diameter, can be made of ceramic, plastic or metal. Advantageously, the metal ball, for example made of stainless steel, makes it possible to obtain a more effective massaging effect. The cap is advantageously made of plastic material which can be covered with a metallic layer, for example made of polypropylene (PP), with an advantageously rigid structure.

The use of a roll-on applicator type device, in addition to the advantages mentioned above, makes it possible to exert physical pressure and/or perform a massage on the application area. This therefore makes it possible to contribute, through mechanical action, to unclogging and relieving congestion in the meibomian glands.

The composition according to the invention has a texture suitable for topical application around the eye, advantageously using such a device.

In a manner known in this technical field, the composition according to the invention can be sterilized before and/or after its packaging in the application device, advantageously the roll-on applicator. The sterilization techniques that can be implemented are well known to those skilled in the art. By way of example, a composition according to the invention can be filtered before it is packaged.

The present invention also relates to the compositions and devices as defined above for prevention, including prevention of recurrence, the management and/or treatment of certain pathologies, in particular swelling of the eyelid.

Particularly targeted in the context of the present invention are pathologies linked to, associated with, resulting from or caused by a dysfunction of the meibomian glands.

It can be mentioned: chalazion, dry eye syndrome, blepharitis and styes. Advantageously, pathologies linked to occlusion (or occlusive dysfunction) of the meibomian glands are targeted, even more advantageously non-infectious such as chalazion.

Indeed, a beneficial effect is reported, even in the absence of therapeutic active ingredient, connected with the formula of the composition according to the invention and its method of administration.

Thus and according to other aspects, the present invention also relates to:
a composition as described above for the management of swelling of the eyelid, advantageously pathologies linked to dysfunction of the meibomian glands, even more advantageously chalazion;
the use of a roll-on applicator comprising a composition as described above for management of swelling of the eyelid, advantageously pathologies linked to dysfunction of the meibomian glands, even more advantageously chalazion;
a method for managing swelling of the eyelid, advantageously a pathology linked to a dysfunction of the meibomian glands, even more advantageously chalazion, by topical application, advantageously on the eyelid, of a composition as described above, advantageously using a roll-on applicator.

The quantity of the composition of the invention, in particular of the three ingredients of interest mentioned above, that is effective in the intended treatment can be determined by standard clinical techniques. Additionally, in vivo and/or in vitro assays, such as those described in the examples below, can potentially be used to help predict optimal dosage ranges. The precise dose to be employed may also depend on the severity of the disease, and should be decided according to the practitioner's judgment and the conditions of each patient.

According to a preferred embodiment, the composition according to the invention is administered daily, with a minimum of twice per day, advantageously in the morning and evening, and up to 6 times per day. It should be noted that apart from the sequences of administration of the composition, the roll-on applicator can be used alone, without releasing the composition, for the advantageous physical effect of massaging, in particular the unclogging of the meibomian glands.

Advantageously, the treatment is implemented as soon as the pathology appears (swelling, redness, irritation, etc. on the lower or upper eyelid), even more advantageously after diagnosis by a doctor or an ophthalmologist. The treatment can last several days or even weeks or even months, advantageously until the symptoms completely disappear. The treatment can also be taken as a preventative measure, particularly against recurrence.

Before applying the composition according to the invention, the area to be treated, advantageously the eyelid, can be:
cleaned using any suitable product such as soapy water or a disinfectant product;
warmed, for example by application of hot water, particularly in the form of compresses.

Before application, the composition according to the invention, advantageously the administration device containing it, are brought to a suitable temperature, for example by friction between the hands.

As illustrated in the examples, the application of the composition according to the invention is advantageously done by massaging using the roll-on applicator, namely back and forth, circular and/or longitudinal movements on and/or along the eyelid to be treated. Advantageously, a longitudinal massage is recommended but a circular massage is possible.

Thus, a composition according to the invention:
provides an easy and pleasant massage function for the eyelids in order to unclog the meibomian large cells causing the chalazion;
provides a function of strengthening the skin barrier function to limit the evaporation of water, and thus protect the eyelid from dehydration;
is compatible with a roll-on tube, allowing a delicate massage of the eyelids by simply rolling the ball on the eyelids to apply the product;
has a soft, viscous, ointment-like texture so it does not flow from the tube at room temperature;
becomes fluid from the start of the massage to ensure the lubrication necessary for massaging the eyelids;
does not leave a greasy and sticky finish on the skin.

The disclosures of each patent, patent application, and publication cited in this application are incorporated by reference in their entirety. Without further description, it is considered that those skilled in the art can, using the following description and illustrative examples, produce and use the compounds of the present invention and implement the claimed methods.

### Embodiment Examples

The invention and the resulting advantages will emerge better from the following embodiment examples. However, these examples have no limiting scope.

The invention is further illustrated using a composition based on castor oil, coconut oil and vaseline packaged in a metal roll-on applicator and in connection with the treatment of chalazion.

### 1/ Composition

A composition according to the invention can have the following formula:

TABLE 1
Composition according to the invention

| Ingredients | Quantity (% by weight) |
|---|---|
| Castor oil (natural non-hydrogenated castor oil) | 50 |
| Coconut oil (natural refined coconut oil) | 30 |
| Vaseline | 12 |
| Glyceryl behenate/Compritol ® (Gattefossé) | 5 |
| Glycerol monostearate/Geleol ® (Gattefossé) | 3 |

### 2/ Characterization of the Composition

### Viscosity:

The composition in Table 1 above has a viscosity value equal to 3800 mPa·s, suitable for application using a roll-on applicator.

### 3/ Use of the Composition

### 3-1 Application Device:

After filtration, the composition in Table 1 is packaged in a roll-on applicator, namely a Polyfoil® tube (capacity for example 10 mL) equipped with a stainless steel metal ball with a diameter of 10 mm. A removable polypropylene (PP) cap is placed on the ball to allow the applicator to be closed.

### 3-2 Application Protocol:

### Dosage

The composition in Table 1 is applied using the roll-on applicator described in point 3-1 as soon as the first signs of chalazion appear.

It is applied on the chalazion and eyelids at least twice a day, in the morning and evening, and up to 6 times a day.

If the chalazion does not disappear after 15 days of application, an ophthalmologist who can prescribe a further treatment should be consulted.

### Recommendations for Use

For maximum effectiveness, it is recommended to warm the eyelids, for example using compresses 5 to 10 minutes before application.

Hands should be washed before use.

If the user wears lenses or glasses, they should be removed.

It is recommended to warm the applicator between hands before use if the outside temperature is below 20° C.

No instrument should be used to open the tube.

The cap should be removed.

The applicator ball is placed on the chalazion and on the skin of the eyelid to be treated.

Gently press the tube and begin a longitudinal massage along the eyelid for 1 minute, then possibly continue with a rotational massage centered on the swelling.

Replace the cap on the tube.

1. A composition comprising:
castor oil, representing 40 to 60% by weight of the composition;
coconut oil, advantageously refined coconut oil, jojoba oil, or avocado oil, representing 20 to 40% by weight of the composition; and
vaseline, representing 5 to 30% by weight of the composition.

2. The composition according to claim 1, characterized in that:
the castor oil represents 45 to 55%, advantageously 50% by weight of the composition; and/or
the coconut oil, advantageously refined coconut oil, represents 25 to 35%, advantageously 30% by weight of the composition; and/or
the vaseline represents 5 to 20%, advantageously 10 to 15%, even more advantageously 12% by weight of the composition.

3. A composition comprising:
castor oil;
coconut oil, advantageously refined coconut oil, jojoba oil, or avocado oil; and
vaseline,
advantageously the composition according to claim 1 or 2,
said composition further comprising glyceryl behenate and/or glycerol monostearate, advantageously glyceryl behenate and glycerol monostearate.

4. The composition according to claim 3, characterized in that:
the glyceryl behenate represents 0.5 to 10%, advantageously 3 to 7%, even more advantageously 5% by weight of the composition; and/or
the glycerol monostearate represents 0.5 to 10%, advantageously 1 to 5%, even more advantageously 3% by weight of the composition.

5. The composition according to any one of claims 1 to 4, characterized in that it is free of preservatives.

6. The composition according to any one of claims 1 to 5, characterized in that it is in the form of a composition intended for topical application, advantageously a gel, an emulsion, an oil, a cream, a balm or an ointment.

7. A composition comprising:
castor oil;
coconut oil, advantageously refined coconut oil, jojoba oil, or avocado oil; and
vaseline,
advantageously a composition according to any one of claims 1 to 6,
for use in the treatment of swelling of the eyelid.

8. A composition comprising:
castor oil;
coconut oil, advantageously refined coconut oil, jojoba oil, or avocado oil; and
vaseline,
advantageously a composition according to any one of claims 1 to 6,
for use in the treatment of obstructive meibomian gland dysfunction.

9. A composition for its use according to claim 8, characterized in that it is used for the treatment of chalazion.

10. A composition for its use according to any one of claims 7 to 9, characterized in that it is free of preservatives.

11. A composition for its use according to any one of claims 7 to 10, characterized in that it is applied topically, advantageously on the eyelid, even more advantageously by longitudinal massage.

12. A roll-on applicator containing a composition comprising:
castor oil;
coconut oil, advantageously refined coconut oil, jojoba oil, or avocado oil; and
vaseline,
advantageously a composition according to any one of claims 1 to 6.

13. The applicator according to claim 12, characterized in that the composition is free of preservatives.

14. The applicator according to claim 12 or 13 for use in the treatment of swelling of the eyelid or obstructive meibomian gland dysfunction, advantageously chalazion.
